(19) United States
(12) Patent Application Publication
(10) Pub. No.: US 2015/0104416 A1
(43) Pub. Date: Apr. 16, 2015

(54) Leukemic Stem Cell Ablation

(71) Applicant: Chemgenex Pharmaceuticals, Inc., Menlo Park, CA (US)

(72) Inventor: Dennis Brown, Menlo Park, CA (US)

(21) Appl. No.: 14/574,989

(22) Filed: Dec. 18, 2014

Related U.S. Application Data
(63) Continuation of application No. 12/330,317, filed on Dec. 8, 2008, now abandoned.
(60) Provisional application No. 61/012,371, filed on Dec. 7, 2007.

(51) Int. Cl. A61K 31/55; A61K 45/06

(52) U.S. Cl. CPC A61K 31/55; A61K 45/06
USPC 424/85.7; 514/214.01; 514/34

(57) ABSTRACT

A method for treating a leukemia patient that is resistant to a thymidine kinase inhibitor (TKI) other than imantinib comprising administering a cephalotaxine to said patient until said patient demonstrates a hematological or cytological response to said leukemia.

### CROSS REFERENCE TO RELATED APPLICATION

This applications claims the benefit under 35 U.S.C. §119(e) of U.S. Provisional Application Ser. No. 61/012,371, filed Dec. 7, 2007, which is incorporated by reference herein.

### FIELD OF THE INVENTION

The invention relates to the use of cephalotaxines to ablate leukemic stem cells in treatment protocols using tyrosine kinase inhibitors (TKIs) and other anti-leukemic agents.

### BACKGROUND OF THE INVENTION

The Abl tyrosine kinase inhibitors (TKIs) imatinib mesylate (IM) and dasatinib, have revolutionized the treatment of Philadelphia-positive (Ph+) leukemia in both chronic myeloid leukemia (CML) and B-cell acute lymphoblastic leukemia (B-ALL) by targeting and disabling the proliferative signal coming from BCR-ABL. However, clinical resistance to these TKIs negates curative results in Ph+ leukemia.

Resistance to tyrosine kinase inhibitor (TKI) is a problem for a subset of patients with CML. Resistance is particularly important for the patients who develop the T315I BCR-ABL kinase domain (KD) mutation which represents approximately 15% of all mutations detected after failure to TKI.

The T315I mutation results in resistance to imatinib mesylate (IM) and the second generation TKIs, including dasatinib (D), nilotinib (N), bosutinib (B) and INNO 406. Currently, no approved therapy has been shown to be efficacious for CML patients harboring the T315I mutation making this an important area of unmet medical need.

### SUMMARY OF THE INVENTION

Methods are disclosed for treating leukemia patients comprising treating the patient with a cephalotaxine followed by treatment with a tyrosine kinase inhibitor (TKI). The cephalotaxine treatment is preferably carried out until the patient demonstrates a hematological or cytological response to the leukemia. If the leukemic cells in a patient develop resistance to the TKI, cephalotaxine treatment is repeated. The cephalotaxine treatment ablates leukemic stem cells and is believed to reduce or eliminate leukemic stem cells including those clonal populations that are resistant to TKI treatment and which would otherwise expand during TKI treatment alone. A clonal population containing the bcr-abl genotype having the T315I mutation is an example of such a population. If necessary, the cephalotaxine treatment is repeated until the patient demonstrates a hematological or cytological response to the leukemia. Thereafter, the patient can be treated with the same or a different TKI.

Current treatment for leukemia, such at CML, call for the treatment of the patient with imatinib (Gleevec). This treatment often results in remission of the disease. However, in many cases resistance to Gleevac arises. One way to treat such patients is to administer the cephalotaxine homoharringtonine (HHT). According to the invention, such TKI resistance patients can be treated with a cephalotaxine which is then followed by treatment with a TKI as described above.

Homoharringtonine (HHT) is a preferred cephalotaxine, although other cephalotaxine analogs can be used. The initial treatment with HHT is preferably about 1.0 to 5.0 mg/m2 HHT per day, more preferably 1.0 to 2.5 mg/m2 HHT per day. The HHT treatment can be for 5 days or more. However, the treatment may be as long as 14 days in a 28 day cycle. In some cases, the amount and/or duration may be less than 2.5 mg/m2 HHT per day and less than 5 days.

The foregoing methods can also be modified so that the treatment with TKI is supplemented with concurrent treatment with a cephalotaxine. In such cases, (1) the amount of cephalotaxine can be lower than that which would be used if administered alone, (2) the time for cephalotaxine treatment can be reduced (e.g. 2-5 days for HHT), or (3) the amount and time cephalotaxine treatment can be reduced. In addition, the amount of TKI can also be lower than if administered alone.

The invention also includes methods to treat leukemic patients who have developed resistance to TKIs other than IM. The treatment is with a cephalotaxine to ablate leukemic cells and leukemic stem cells that have acquired such resistance. Such patients may be contemporaneously treated with a TKI or subsequently treated with a TKI after the patient demonstrates a hematological or cytological response to the leukemia.

In addition, other anti-leukemia agent can be administered to the patient before, during, or after administration of cephalotaxine or TKI. Such additional treatment includes the use of inhibitors of SRC-kinases.

### BRIEF DESCRIPTION OF THE DRAWINGS

FIGS. 1A, 1B and 1C demonstrate that HHT inhibits the proliferation of myeloid and lymphoid cells. In FIG. 1A the number of viable cells at the indicated drug concentrations was determined by trypan blue. In FIG. 1B the expression of ABL and MCL-1 in K562 (myeloid) cells is inhibited by HHT. In FIG. 1C the expression of ABL in lymphoid cells with BCR-ABL or the BCR-ABL T315I mutation is inhibited by HHT.

FIGS. 2A, 2B and 2C demonstrates that HHT reduces circulating leukemic (GFP+) cells, reduces spleen weight and improves survival in mice with BCR-ABL-WT-induced CML. FIG. 2A is a FACS analysis of circulating GFP+ cells in mice with BCR-ABL-WT-induced CML. The FACS plot shows the cell distribution for mice treated with placebo or HHT. The number of circulating leukemic cells (calculated as percentage of Gr-1+GFP+cells×white blood cell count) in mice with BCR-ABL-WT-induced CML treated with placebo or HHT for 4 days was determined on day 12 after transplantation. FIG. 2B depicts a bar graph for the leukemic cells as well as a bar graph for spleen weight of the mice treated with placebo or HHT. FIG. 2C demonstrates survival of CML mice treated with HHT as compared to those treated with placebo.

FIGS. 3A and 3B demonstrate that HHT improves survival of mice with BCR-ABL-T315I-induced CML. In FIG. 3A, the number of circulating leukemic cells (calculated as percentage of Gr-1+GFP+cells×white blood cell count) in mice with BCR-ABL-T315I-induced CML treated with placebo or HHT was determined on day 14 after transplantation. FIG. 3B demonstrates that treatment with the HHT prolonged survival of BCR-ABL-T315I induced CML mice.

FIGS. 4A and 4B demonstrate that HHT improves survival of mice with BCR-ABL-induced B-ALL. FIG. 4A is a FACS analysis evaluation of the leukemic process in HHT or placebo treated B-ALL mice after 4 days or 10 days. FIG. 4B shows that treatment with the HHT prolonged survival of BCR-ABL induced B-ALL mice.

FIGS. 5A, 5B and 5C demonstrate that HHT inhibits the survival of leukemic stem cells. FIG. 5A is a FACS analysis of bone marrow cells isolated from mice with BCR-ABL-WT-induced CML were cultured in vitro (5×106 cells/6 cm tissue culture plate) in the presence of different doses of HHT for 6 days (changing the stem cell medium containing placebo or HHT at day 3) followed by FACS analysis of leukemia stem cells (GFP+Lin-c-Kit+Sca-1+). FIG. 5B depicts bar graphs for the leukemic cells and leukemic stem cells. FIG. 5C shows bar graphs for leukemic cells and leukemic stem cells from mice with BCR-ABL-WT-induced CML treated with a placebo, imatinib (100 mg/kg, twice a day by gavage), and HHT (0.5 mg/kg, once every day by gavage), respectively, for 4 days beginning at day 8 after transplantation. Bone marrow cells were isolated from the treated CML mice, and leukemia stem cells were analyzed by FACS. The numbers of cells represent total leukemia stem cells in average from femur and tibia of each treated CML mouse.

FIG. 6 depicts the proposed mechanism of action for HHT.

FIG. 7 shows the progression free survival of patients treated with HHT after acquiring resistance to various TKIs as a funcion of time (months). Of those in chronic phase (CP), 80% survived after 1 year and 70% survived after 2 years. Of those in accelerated phase (AP), 25% survived after one year. Of those in blast phase (BP), 44% survived after 6 months. Events were defined as death, study withdrawal due to AE or disease progression.

FIG. 8 depicts T115I expression in chronic phase patients after treatment with HHT. The T315I mutated clone is rapidly and substantially reduced in CP patients. In 64% of patients, the T315I clone is reduced to below the limits of detection.

FIG. 9 shows the chemical structure of cephalotaxines.

FIG. 10 shows the chemical structure of HHT.

### DETAILED DESCRIPTION

The invention is based, in part, upon the discovery that the cephalotaxine HHT ablates leukemic stem cells, including those that have developed resistance to TKI treatment. This discovery provides for anti-stem cell treatment protocols wherein a cephalotaxine is administered to a leukemia patient to ablate leukemic stem cells followed by treatment with a TKI. If resistance to the TKI develops, treatment with the same or a different cephalotaxine ablates the leukemic stem cells, including TKI resistant leukemic stem cells. Thereafter, treatment with the same or a different TKI can be resumed. This cycle can be repeated as necessary necessary to improve the hematological and cytogenetic responses.

The invention is also based, in part, on the discovery that leukemic patients who have developed resistance to TKIs other than IM can be treated with a cephalotaxine to ablate leukemic cells and leukemic stem cells that have acquired such resistance. Such patients may be contemporaneously treated with a TKI or subsequently treated with a TKI after the patient demonstrates a hematological or cytological response to the leukemia. Repeated cycles of such therapies may be employed to induce durable progression free responses.

Other anti-leukemia agents can be administered to the patient before, during, or after administration of cephalotaxine or TKI. Such additional treatment includes the use of inhibitors of SRC-kinases, aurora kinases, immunomodulators such as interferon-alpha, conventional hemotherapeutics such as hydroxyurea, ara-C, doxorubicin and the like.

As used herein leukemia refers to chronic myelogenous leukemia (CML), acute myelogenous leukemia (AML), acute lymphocytic leukemia (ALL), chronic lymphocytic leukemia (CLL) and acute promyelocytic leukemia (APL). Leukemia also includes pre-leukemic syndromes such myelodysplastic syndrome.

As used herein, TKI refers to any thymidine kinase inhibitor. Examples of TKIs include to imatinib mesylate (IM) and second generation TKIs, including dasatinib (D), nilotinib (N), bosutinib (B) and INNO 406.

As used herein, a leukemic stem cell refers to a pluripotent stem cell characterized by genetic transformation resulting in unregulated cell division. For example in CML, the BCR-ABL fusion gene (Philadelphia chromosome).

As used herein “ablation” refers to the partial or complete removal of luekemic stem cells and their progeny from a patient. Since many cephalotaxines are know to have deleterious effects on normal leukocytes, cephalotaxine treatment to ablate leukemic stem cells may be limited by the extent of the patient's overall hematological or cytological response. However, the ablation of a significant portion of the leukemic cell population and the stem cells that form them provides an opportunity for TKI treatment that can be conducted at lower dose levels and/or for a longer time period before the onset of TKI resistance.

As used herein, a cytological response to treatment with cephalotaxine and/or TKI is a response that occurs in the bone marrow rather than just in the peripheral blood. There are at least three cytological responses: (1) a cytogenetic response (CR); (2) a major cytogenetic response (MCR); and a complete cytogenetic response (CCR). Determination of such responses is based on the measurement of the number of peripheral blood and/or bone marrow cells having a marker that is associated with a particular leukemia. Such markers include cell surface antigens, aberrant proteins, and genetic modifications. A cytogenetic response occurs if the number or percentage of cells with such a marker decreases during treatment. A major cytogenetic response occurs if the number or percentage of such cells falls below 35%. A complete cytogenetic response occurs when no cells containing the marker are detected. For example, CML is characterized by the Ph+ chromosome. A cytogenetic response has occurred if the number of Ph+ chromosomes decreases at all during treatment. If the Ph+ percentage drops to 35% or less, it is considered a major cytogenetic response; 0% Ph+ is a complete cytogenetic response.

As used herein, a hematological response occurs when there is a change in the white blood cell count of a patient following treatment with cephalotaxine and/or TKI. The change in white blood cell count can be in the peripheral blood and the bone marrow although the change may be observed only in the peripheral blood. The response can be a partial reduction in white cell count or complete reduction to normal values (e.g. 10,000-12,000 cells per ml.)

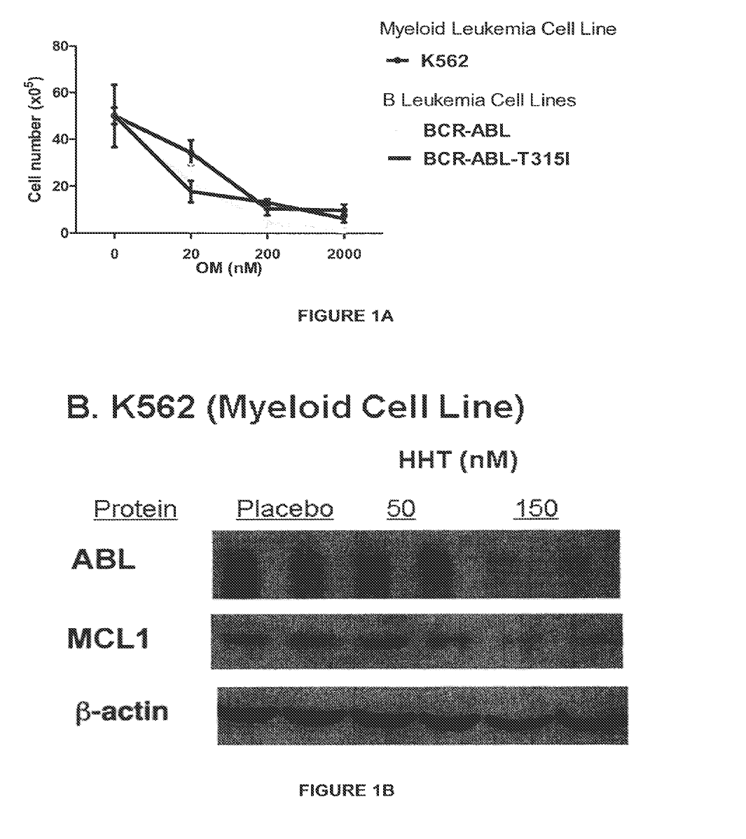

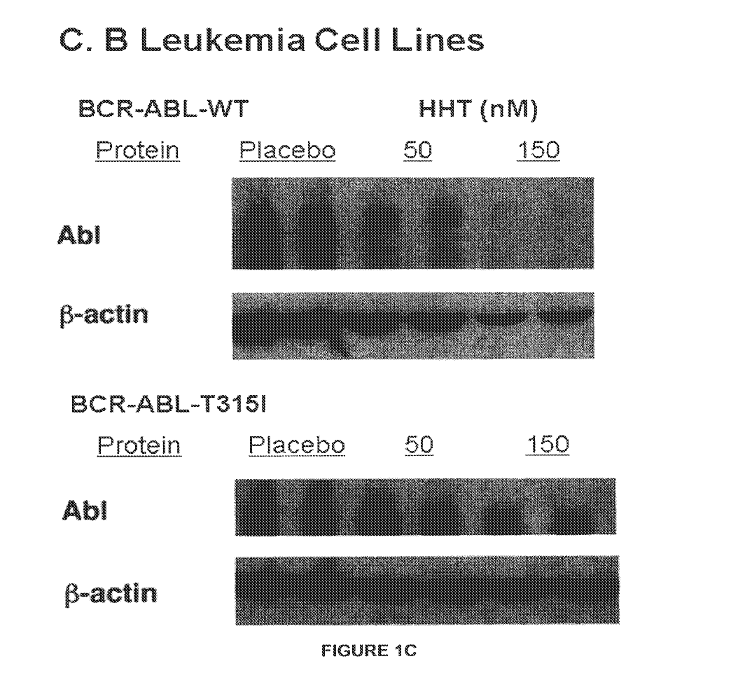

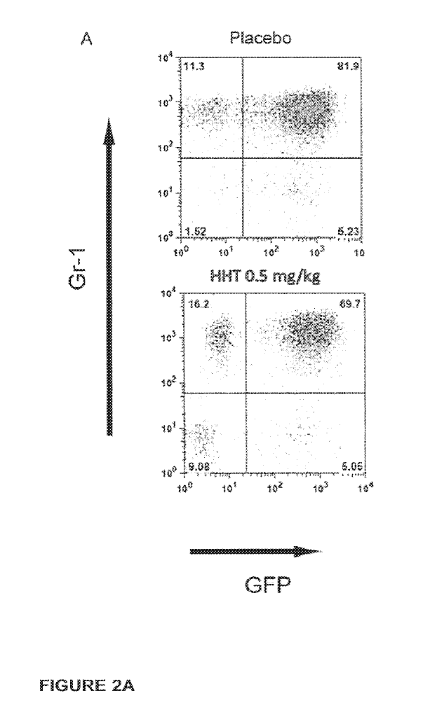

As used herein, the term cephalotaxine includes all members of that chemical family including alkaloid derivatives of the Chinese evergreen, Cephalotaxus fortunei Hook and other related species, such as Cepholotaxus sinensis Li, C. hainanensis and C. wilsoniana, including C. oliveri mast and C. harringtonia (Powell, R. G., (1972) J. Pharm Sci., 61(8):1227-1230) and analogs thereof. The cephalotaxine family is defined by the chemical structure as set forth in FIG. 1.

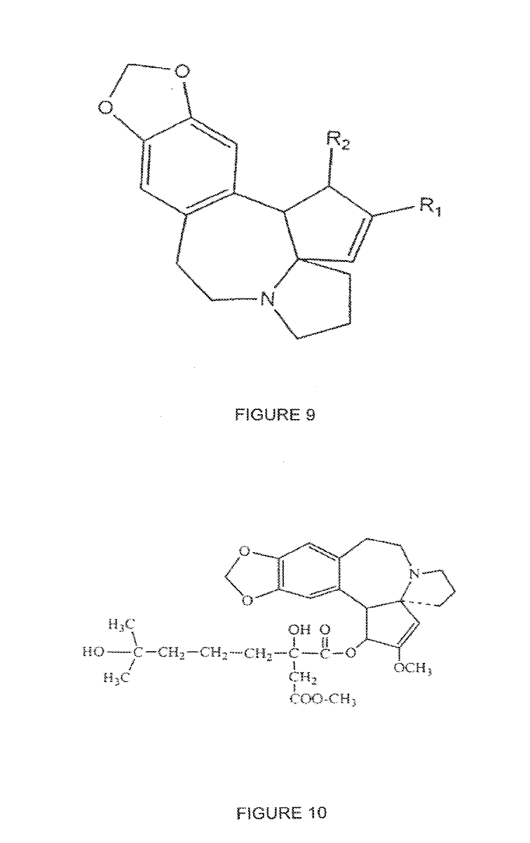

A cephalotaxine analog is further defined but not limited to the structure depicted in FIG. 9, having substituent or substitute groups at R1 and R2. Examples of R1 and/or R2 include esters, including herringtonine, isoharringtonine, homoharringtonine, deoxyharringtonine, acetylcephalotaxine and the like. Table I lists structures of R1 and R2 for some of these analogs. R1 and R2 substitutions are typically employed to improve biological activity, pharmaceutical attributes such as bioavailability or stability, or decrease toxicity. In one embodiment, R1 and/or R2 include alkyl substitutions (e.g., methyl, ethyl, propyl etc.). In another embodiment, R1 and/or R2 include esters (e.g., methoxy, ethoxy, butoxy, etc.). R1 and R2 are not limited to the above examples, however, in the scope of this invention.

TABLE I

| compound | R1 | R2 |
|---|---|---|
| isoharringtonine | —OCH₃ | 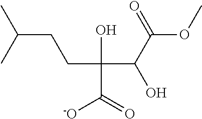 |
| harringtonine | —OCH₃ | 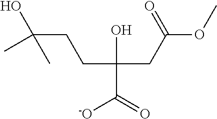 |
| acetyl- cephalotaxine | —OCH₃ | 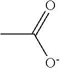 |
| homo- harringtonine | —OCH₃ | 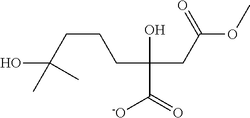 |

A specific example of cephalotaxine is homoharringtonine which is the butanediocate ester of cephalotaxine, 4-methyl-2-hydroxy-2-(4-hydroxy-4-methyl pentyl). Its chemical structure is set forth in FIG. 10.

The cephalotaxine formulations include those suitable for oral or parenteral (including subcutaneous, intramuscular, intravenous) administration. The formulations may conveniently be presented in unit dosage form and may be prepared by conventional pharmaceutical techniques. Such techniques include the step of bringing the active ingredient into association with the pharmaceutical carrier(s) or excipient(s). In general, the formulations are prepared by uniformly and intimately bringing into association the active ingredient with liquid carriers or finely divided solid carriers or both, and then, if necessary, shaping the product.

Cephalotaxine formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein for minutes to hours to days.

Preferred oral formulations are disclosed in PCT US2008/60251.

When oral formulations are used, the oral cephalotaxine dosage form preferably is administered to a host in the range of 0.05-5.0 mg/m². In a preferred embodiment, the cephalotaxine is administered to a host in the range of 0.1 to 3.0 mg/m². In a further preferred embodiment, the cephalotaxine is administered to a host in the range of 0.1-1.0 mg/m² and is administered once or multiple times per day.

Cephalotaxine formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described. The unit parenteral dose may contain 1-20 mg of cephalotaxine, more preferred 1-5 mg per unit dose.

Cephalotaxine preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations may include other agents conventional in the art having regard to the type of formulation in question, for example, those suitable for oral administration may include flavoring agents.

### EXAMPLES

Homoharringtonine (Omacetaxine mepesuccinate—USAN/INN designation) (HHT) has shown significant clinical activity in CML in combination with IM and alone for patients failing IM. Utilizing a BCR-ABL-expressing leukemic stem cells (Lin-c-Kit+Sca-1+), K562 myeloid cells and B-cells containing BCR-ABL-WT or BCR-ABL-T315I, HHT was tested for efficacy both in vitro and in CML mice. Not only did HHT inhibit the proliferation of all leukemic cell lines tested, but HHT also provided a significant survival benefit to mice with CML and B-ALL. Additionally, HHT inhibited the expression of the anti-apoptotic protein, Mcl-1, in K562 cells. Inhibiting this protein may be a key target for HHT. In summary, HHT has an inhibitory activity against CML stem cells, and is highly effective in treating CML and B-ALL induced by BCR-ABL in mice.

### Example 1

This example demonstrates that HHT inhibits the proliferation of myeloid and lymphoid cells. Antibodies for Western Blot analysis against c-ABL, Mcl-1 and β-actin were purchased from Santa Cruz Biothechology (Santa Cruz, Calif.). Protein lysates were prepared by lysing cells in RIPA buffer and immunoprecipitation. The retroviral vector MSCV-IRES-eGFP carrying the BCR-ABL cDNA was used to make virus stock for bone marrow transduction/transplantation. Four- to 10-week-old wild-type BABL/c or C57BL/6 (The Jackson Laboratory) and homozygous SRC triple gene knockout (Lyn−/− Hck−/−Fgr−/−) mice were used for leukemogenesis experiments. HHT (ChemGenex Pharmaceuticals, Inc, CA) was dissolved in accompanying diluent to a stock concentration of 1 mg/ml. Further dilutions were made to working concentrations using media or water.

FIG. 1A shows the number of viable cells at the indicated drug concentration of HHT (OM) as determined by trypan blue. As demonstrated in FIG. 1B, the expression of ABL and MCL-1 in K562 (myeloid) cells is inhibited by HHT. As indicated in FIG. 1C, the expression of ABL in lymphoid cells with BCR-ABL or the BCR-ABL T315I mutation is inhibited by HHT.

### Example 2

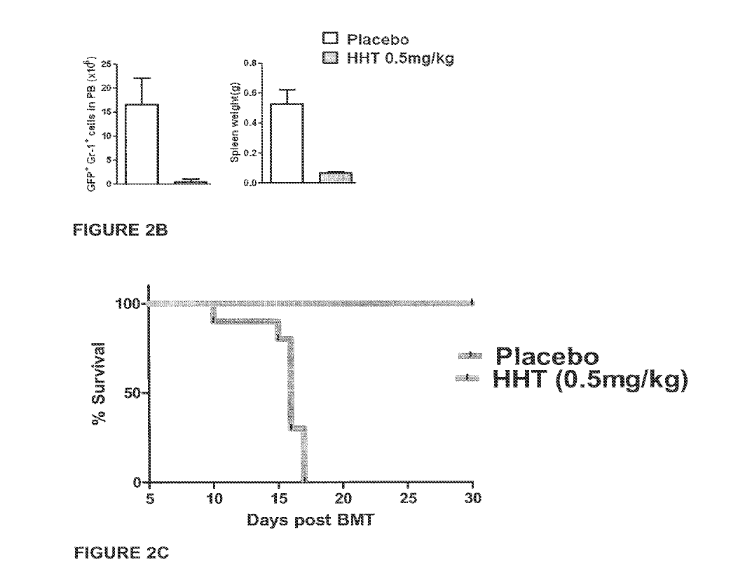

This example demonstrates that HHT reduces circulating leukemic cells, reduces spleen weight and improves survival in mice with BCR-ABL-WT-induced CML. FIG. 2A is a FACS analysis of circulating leukemic GFP+ cells in mice with BCR-ABL-WT-induced CML. This FACS plot shows the cell distribution for mice treated with placebo or HHT (0.5 mg/kg). The number of circulating leukemic cells (calculated as percentage of Gr-1+GFP+cells×white blood cell count) in mice with BCR-ABL-WT-induced CML treated with placebo or HHT for 4 days was determined on day 12 after transplantation. FIG. 2B depicts a bar graph for the leukemic cells as well as a bar graph for spleen weight of the mice treated with placebo or HHT. FIG. 2C demonstrates survival of CML mice treated with HHT as compared to those treated with placebo. HHT therefore significantly reduces the number of circulating leukemic cells and the size of the spleen in CML mice. HHT also significantly increased the survival of the CML mice.

### Example 3

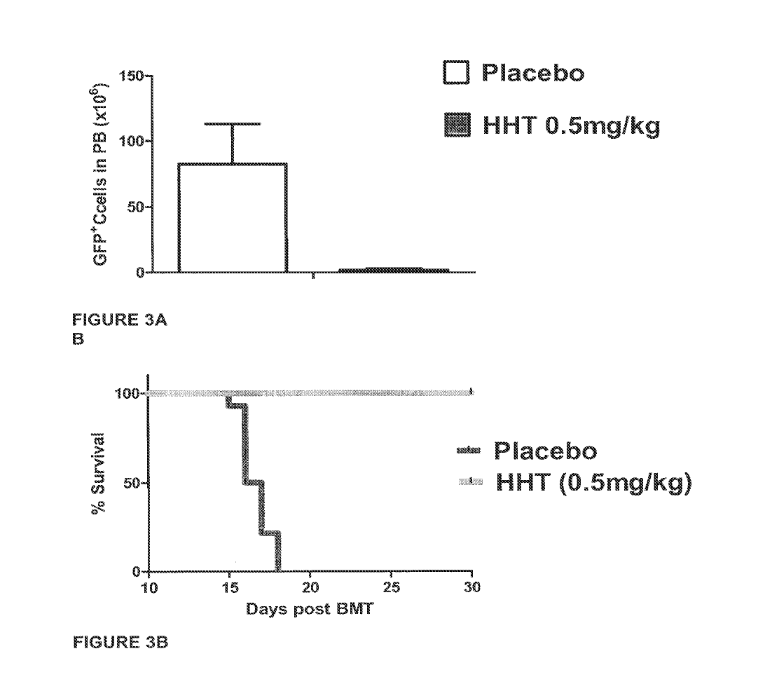

This example demonstrates that HHT improves survival of mice with BCR-ABL-T315I-induced CML. The number of circulating leukemic cells (calculated as percentage of Gr-1+GFP+cells×white blood cell count) in mice with BCR-ABL-T315I-induced CML treated with placebo or HHT (0.5 mg/kg) was determined on day 14 after transplantation. HHT significantly reduced the number of circulating leukemic cells as compared to the placebo treated group. FIG. 3B demonstrates that treatment with the HHT also prolonged survival of BCR-ABL-T315I induced CML mice.

### Example 4

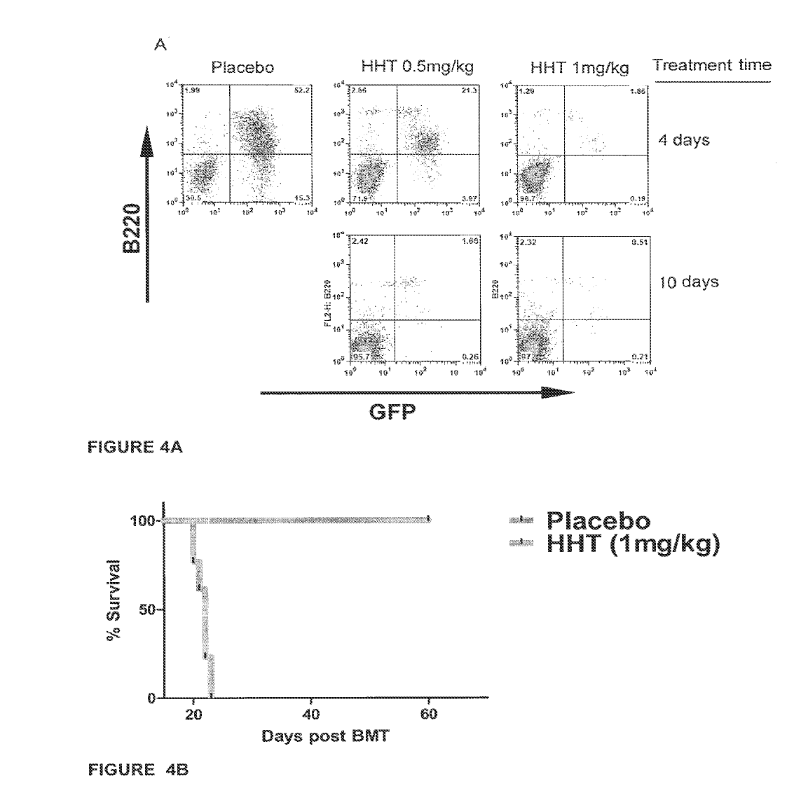

This example demonstrates that HHT improves survival of mice with BCR-ABL-induced B-ALL. FIG. 4A is a FACS analysis evaluation of the leukemic process in HHT or placebo treated B-ALL mice after 4 days or 10 days. As can be seen, there is a significant reduction in leukemic cells as a function of treatment time and HHT dosage. FIG. 4B confirms that treatment with the HHT (1 mg/kg) prolonged survival of BCR-ABL induced B-ALL mice.

### Example 5

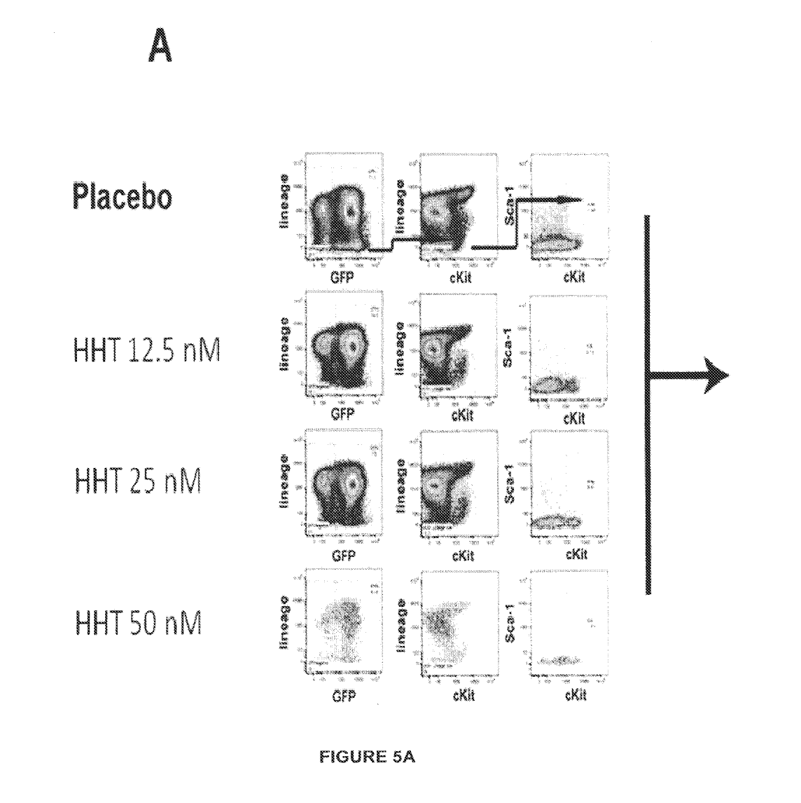

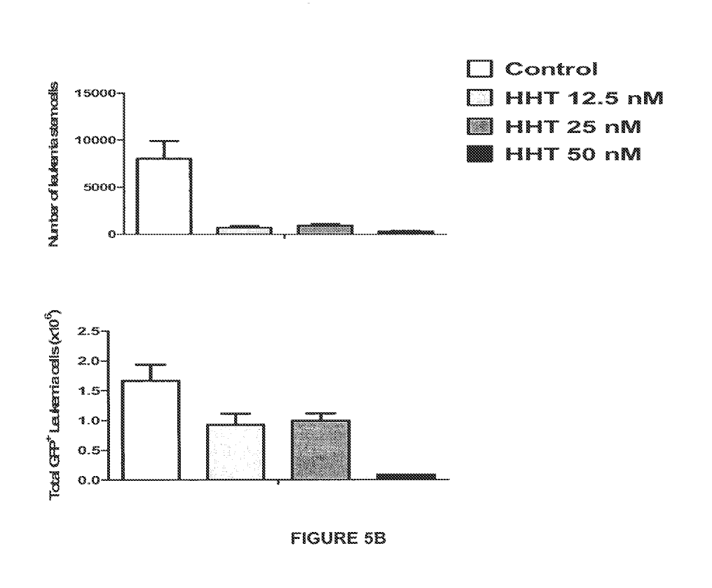

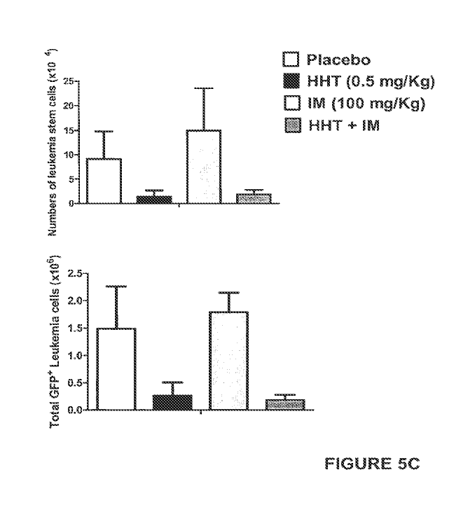

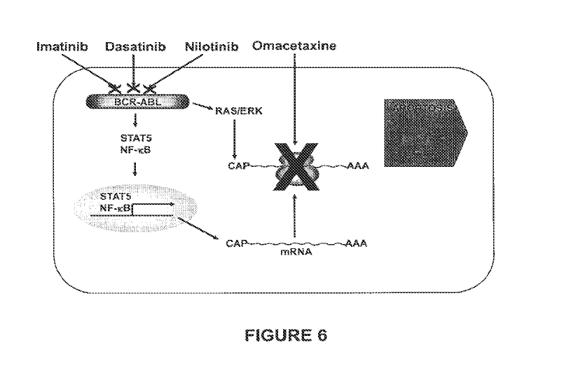

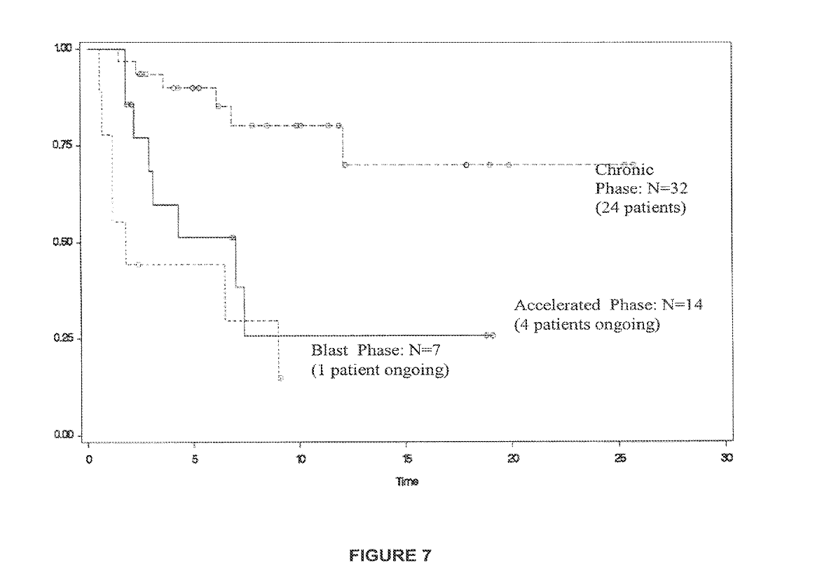

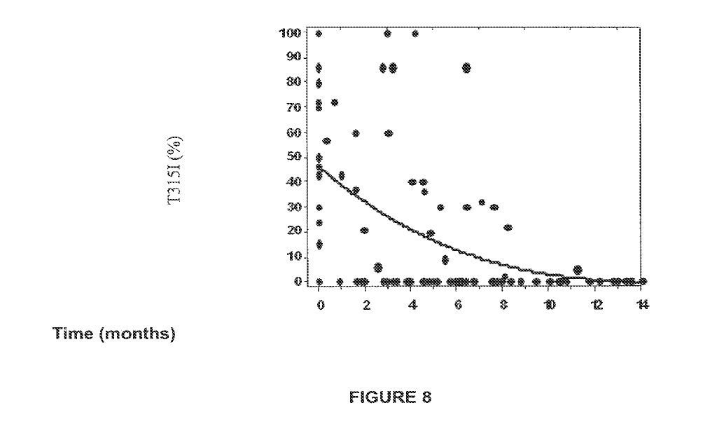

This example demonstrates that HHT inhibits the survival of leukemic stem cells. FIG. 5A is a FACS analysis of bone marrow cells isolated from mice with BCR-ABL-WT-induced CML that were cultured in vitro (5×106 cells/6 cm tissue culture plate) in the presence of different doses of HHT for 6 days (changing the stem cell medium containing placebo or HHT at day 3) followed by FACS analysis of leukemia stem cells (GFP+Lin-c-Kit+Sca-1+). FIG. 5B depicts bar graphs for the leukemic cells and leukemic stem cells. These results clearly indicate that HHT reduces not only the number of leukemic cells but also the number of leukemic stem cells.

FIG. 5C shows bar graphs for leukemic cells and leukemic stem cells from mice with BCR-ABL-WT-induced CML treated with a placebo, imatinib (100 mg/kg, twice a day by gavage), and HHT (0.5 mg/kg, once every day by gavage), respectively, for 4 days beginning at day 8 after transplantation. Imatinib was dissolved in water directly at a concentration of 10 mg/ml. The drugs were given orally in a volume of <0.5 ml by gavage twice a day, at 0.5 mg or 1.0 mg per kilogram of body weight for HHT and 100 mg per kilogram of body weight per dose of imatinib, beginning at 8 days after BM transplantation and continuing until the morbidity or death of the leukemic mice.

Bone marrow cells were isolated from the treated CML mice, and leukemia stem cells were analyzed by FACS. The numbers of cells represent total leukemia stem cells in average from femur and tibia of each treated CML mouse. These results indicate that treatment of CML mice with HHT alone or in combination with imatinib reduces not only the number of leukemic cells but also the number of leukemic stem cells.

The results of the experiments as set forth in Examples 1-5 demonstrate that: (a) HHT inhibits the proliferation of (i) BCR-ABL-expressing leukemic stem cells, (ii) K562 myeloid cells and (iii) B-cells containing either BCR-ABL or BCR-ABL-T315I; (b) HHT inhibits the expression of ABL and the anti-apoptotic protein, Mcl-1, in K562 cells; (c) unlike imatinib, HHT reduces the number of leukemic stem cells in mice with BCR-ABL-WT-induced CML; and (d) HHT provides a significant survival benefit to mice with CML and B-ALL and may circumvent the need to target tyrosine kinases.

### Example 6

This example summarizes human clinical trials using HHT to treat patients with IM-resistant T315I+CML.

### Mechanism of Action of Omacetaxine (HHT)

The mechanism of action of HHT is independent from TK inhibition. HHTs mechanism of action is independent of Bcr-Abl's mutational status which enables it to inhibit CML clones no longer controlled by TKIs. HHT transiently inhibits protein synthesis, with an effect primarily on short-lived proteins, including Bcr-Abl and the anti-apoptotic protein Mcl-1 resulting in enhanced cell death

### Trial Design

A multicenter open-label phase 2/3 study evaluating safety and efficacy of omacetaxine administered subcutaneously (SC) in patients with IM-resistant T315I+CML in all phases of the disease is being conducted.

### BCR-ABL Transcript Levels

Presence of T315I is confirmed at study entry at one of two reference labs (University of Texas MD Anderson Cancer Center or University of Heidelberg, Mediz\grave{l}nĺsche Fakultät Mannheim).

Peripheral blood BCR-ABL transcript levels are determined during omacetaxine therapy by quantitative real-time polymerase chain reaction (qRT-PCR) and BCR-ABL KD mutation analyses.

### Treatment

Induction phase—1.25 mg/m2 twice daily SC for 14 consecutive days every 28 days until complete hematologic response (CHR) or hematologic improvement has been achieved.

Maintenance therapy—1.25 mg/m2 twice daily SC for 7 days every 28 days for up to 24 months.

Doses were adjusted to maintain adequate WBC and platelet control by increasing or decreasing the number of days of administration of omacetaxine.

### Results

TABLE 2
Baseline Characteristics

| | Chronic | Accelerated | Blast | |
|---|---|---|---|---|
| | Phase | Phase | Phase | Total |
| Characteristic | N = 32 | N = 14 | N = 9 | N = 55 |
| Age - years | | | | |
| Median | 61 | 65 | 51 | 58 |
| Range | 26-84 | 30-84 | 19-62 | 19-84 |
| Sex - number (%) | | | | |
| Male | 22 (69) | 10 (71) | 7 (78) | 39 (71) |
| Female | 10 (31) | 4 (28) | 2 (22) | 16 (29) |
| Duration of | | | | |
| Disease- months | | | | |
| Median | 49 | 100 | 47 | 58 |
| Range | 13-190 | 18-289 | 5-71 | 5-289 |
| Prior TKI therapy | | | | |
| IM only | 9 (28) | 1 (7) | 1 (11) | 11 (20) |
| 2 TKIs | 16 (50) | 7 (50) | 6 (67) | 29 (53) |
| 3 or more TKIs | 7 (22) | 6 (43) | 2 (22) | 15 (27) |

TABLE 3
Hematologic and Cytogenetic Response

| | Chronic | Accelerated | Blast |
|---|---|---|---|
| Response | Phase | Phase | Phase |
| Number (%) | N = 25 | N = 11 | N = 8 |
| Hematologic Response | | | |
| Overall | 20 (80) | 5 (45) | 1 (13) |
| Complete Hematologic Response | 20 (80) | 2 (18) | — |
| (CHR) | | | |
| Hematologic Improvement | NA | 1 (9) | — |
| (HI) | | | |
| Return to Chronic Phase | NA | 2 (18) | 1 (13) |
| (RCP) | | | |
| Cytogenetic Response | | | |
| Overall | 7 (28) | 1 (9) | — |
| Major* | 5 (20) | — | — |
| Complete | 4 (16) | — | — |
| Partial | 1 (4) | — | — |
| Minimal | 2 (8) | 1 (9) | — |
| Molecular Response | | | |
| Major | 2 (8) | — | — |

*One complete and one partial cytogenetic response are unconfirmed.

### Transcript Mutations: Data Available for 13 Patients

The T315I mutated BCR-ABL transcripts have been reduced below the detection level in 38% (5/13) of evaluable patients (1 AP patient achieved a PHR; 4 CP patients achieved CHR).

TABLE 4
Treatment Outcomes: Data available on 21 patients

| | | | | | | T315I % | T315I % | Time to | | |
|---|---|---|---|---|---|---|---|---|---|---|
| | | | | | | of BCR- | Decrease | Response (No. | Duration of | |
| Patient | Disease | Original Date | CHR at | Prior | Best | ABL at | from | of Treatment | Response | |
| ID | Phase | of Diagnosis | Baseline | Treatment | Response | Baseline | Baseline | Cycles) | (Months) | Outcome |
| 1 | CP | 3/04 | Y | IM | MiCyR | 50 | 100 | 2 | 12+ | Ongoing |
| 2 | CP | 7/02 | N | IM, N | CHR | 20 | 100 | 2 | 12+ | Ongoing |
| 3 | CP | 5/99 | Y | IM, N, | CCyR | 40 | 0 | 3 | 4+ | Ongoing |
| | | | | interferon | | | | | | |
| | | | | alpha | | | | | | |
| 4 | CP | 4/99 | N | IM, D, ara-c, | CHR | 30 | 100 | 3 | 4+ | Ongoing |
| | | | | HHT | | | | | | |
| | | | | Interferon | | | | | | |
| | | | | alpha | | | | | | |
| 5 | CP | 4/01 | N | IM, D | CHR | 72 | * | 2 | 4+ | Ongoing |
| 6 | CP | 4/04 | N | IM, | CHR | 100 | 100 | 5 | 1+ | Ongoing |
| | | | | tipifarnib, | | | | | | |
| | | | | B | | | | | | |
| 7 | CP | 9/05 | N | IM | CHR | 70 | * | 1 | 3+ | Ongoing |
| 8 | CP | 8/03 | N | IM, D, N | Hi | 50 | 17 | 1 | 3+ | Ongoing |
| 9 | CP | 12/05 | Y | IM | CCyR | 80 | * | 3 | 4+ | Ongoing |
| 10 | CP | 1/06 | N | IM, D | None | 50 | * | N/A | N/A | Ongoing |
| 11 | CP | 1998 | N/A | IM, D, ara-c, | PD | * | * | N/A | N/A | PD |
| | | | | Interferon | | | | | | |
| | | | | alpha | | | | | | |
| 12 | AP | 1/01 | N/A | IM, D, N, | Hi | 100 | 0 | 2 | 5 | PD |
| | | | | MK-0457, | | | | | | |
| | | | | KW2449, | | | | | | |
| | | | | ara-c, | | | | | | |
| | | | | Interferon | | | | | | |
| | | | | alpha | | | | | | |
| 13 | AP | 1999 | N/A | IM, D | None | 42 | 0 | N/A | N/A | Ongoing |
| 14 | AP | 6/04 | N/A | IM, D, N | CHR | * | * | 3 | 2+ | Ongoing |
| | | | | | and | | | | | |
| | | | | | MiCyR | | | | | |
| 15 | AP | 12/05 | N/A | IM, N | PHR | 36 | 100 | 1 | 1.5 | PD |
| 16 | BP | 10/06 | N/A | IM, D, N, | PD | 50 | 0 | N/A | N/A | PD |
| | | | | ara-c, | | | | | | |
| | | | | Multiple | | | | | | |
| | | | | chx. | | | | | | |
| | | | | regimens | | | | | | |
| 17 | BP | 3/03 | N/A | IM, D, ara-c. | PD | 50 | 0 | N/A | N/A | PD |
| | | | | Multiple | | | | | | |
| | | | | chx. | | | | | | |
| | | | | regimens | | | | | | |
| 18 | BP | 6/01 | N/A | IM, D | PD | 98 | 0 | N/A | N/A | Death |
| 19 | BP | 2/07 | N/A | IM, D, ara-c, | PD | 69 | 0 | N/A | N/A | Death |
| | | | | MK-0457, | | | | | | |
| | | | | idarubicin | | | | | | |
| 20 | BP | 11/02 | N/A | IM, D, N, | Hi | * | * | 3 | 1+ | Ongoing |
| | | | | ara-c, | | | | | | |
| | | | | LBH589, | | | | | | |
| 21 | BP | 1/07 | N/A | IM | PD | * | * | N/A | N/A | Death |

*data pending
IM—imatinib; D—dasatinib; N—nilotinib, PD—progressive disease; CCyR—complete cytogenetic response; MiCyR—minor cytogenetic response; CHR—complete hematoiogic response; PHR—partial hematologic response; HI—hematologic improvement; chx.—chemotherapy.

TABLE 5
Duration of Response

| | Chronic | Accelerated | Blast |
|---|---|---|---|
| Median Duration of Response | Phase | Phase | Phase |
| Months (range) | N = 20 | N = 5 | N = 1 |
| Hematologic Response | | | |
| CHR | 11.5 (3.5-25.4+) | 9.6 (8.3-10.9+) | — |
| HI | NA | 2.8 | — |
| RCP | NA | 2.0 (2.0-2.0+) | 3.4 |
| Cytogenetic Response | | | |
| Complete | 4.8 (0.3-9.7+) | — | — |
| Partial | 4.2 | — | — |
| Minimal | 4.0 (3.9-20.8+) | 2.3 | — |

### Safety and Tolerability

Dose delays occurred in 75% of induction and 80% of maintenance cycles. Grade 1/2 injection site erythema and pain where reported in 6 (23%) and 3 (13%) of patients, respectively.

TABLE 6
Grade 3/4 Toxicities*

| | Chronic | Accelerated | Blast | |
|---|---|---|---|---|
| Adverse Event | Phase | Phase | Phase | Total |
| number (percent) | N = 12 | N = 5 | N = 6 | N = 23 |
| Hematologic | | | | |
| Thrombocytopenia | 8 (67) | 3 (60) | 1 (17) | 12 (52) |
| Anemia | 7 (58) | 2 (40) | 1 (17) | 10 (43) |
| Neutropenia | 6 (50) | 2 (40) | 2 (33) | 10 (43) |
| Febrile neutropenia | 1 (9) | 2 (40) | 2 (40) | 5 (22) |
| Pancytopenia | 1 (9) | 2 (40) | 0 | 3 (13) |
| Gastrointestinal | | | | |
| Diarrhea | 2 (17) | 1 (20) | 1 (17) | 4 (17) |

Includes all events considered related, probably related, possibly related, or with unknown relationship to omacetaxine treatment. Events listed in the table are included if occurrence was ≧5% of the total population (at least 1 of 23 patients).

TABLE 7
Treatment-Related Serious Adverse Events

| | Chronic | Accelerated | Blast | |
|---|---|---|---|---|
| Event | Phase | Phase | Phase | Total |
| Number (percent) | N = 12 | N = 6 | N = 6 | N = 23 |
| Neutropenic Fever | 1 (9) | 1 (9) | 1 (9) | 3 (13) |
| Thrombocytopenia | 1 (9) | 0 | 0 | 1 (4) |
| Acute Coronary | 1 (9) | 0 | 0 | 1 (4) |
| Syndrome* | | | | |

*This event was considered secondary to omacetaxine induced anemia

Deaths on study: 3 deaths occurred in BP patients with progressive disease, none were considered related to the study drug.

These results demonstrate that: (a) subcutaneously administered omacetaxine is generally well tolerated with myelosuppression as the most common side effect; (b) myelosuppression is usually transient and reversible, and rarely results in serious clinical complications; (c) Omacetaxine therapy has led to the complete elimination of the T315I clone in a number of heavily-pretreated patients with IM-resistant T315I+CML; (d) Omacetaxine therapy has demonstrated complete hematologic and cytogenetic responses with duration up to one year.

1-19. (canceled)

20. A method for treating a patient with leukemia that is resistant to dasatinib, said method comprising:
administering omacetaxine mepesuccinate to said patient until said patient demonstrates a hematological or cytological response.

21. The method of claim 20 further comprising administering an additional anti-leukemia agent.

22. The method of claim 21 wherein said additional anti-leukemia agent is selected from tyrosine kinase inhibitors, SRC-kinase inhibitors, aurora kinase inhibitors, interferon alpha, hydroxyurea, ara-C or doxorubicin.

23. The method of claim 21 wherein omceataxine mepesuccinate is administered before said additional anti-leukemia agent.

24. The method of claim 21 wherein omacetaxine mepesuccinate is administered after said additional anti-leukemia agent.

25. The method of claim 21 wherein omacetaxine mepesuccinate is administered contemporaneously with said additional anti-leukemia agent.

26. The method of claim 25 wherein said additional anti-leukemia agent is a tyrosine kinase inhibitor, SRC-kinase inhibitor, or aurora kinase inhibitor.

27. A method for treating a patient with leukemia that is resistant to bosutinib, said method comprising:
(a) administering omacetaxine mepesuccinate to said patient until said patient demonstrates a hematological or cytological response.

28. The method of claim 27 further comprising administering an additional anti-leukemia agent.

29. The method of claim 28 wherein said additional anti-leukemia agent is selected from tyrosine kinase inhibitors, SRC-kinase inhibitors, aurora kinase inhibitors, interferon alpha, hydroxyurea, ara-C or doxorubicin.

30. The method of claim 28 wherein omceataxine mepesuccinate is administered before said additional anti-leukemia agent.

31. The method of claim 28 wherein omacetaxine mepesuccinate is administered after said additional anti-leukemia agent.

32. The method of claim 28 wherein omacetaxine mepesuccinate is administered contemporaneously with said additional anti-leukemia agent.

33. The method of claim 32 wherein said additional anti-leukemia agent is a tyrosine kinase inhibitor, SRC-kinase inhibitor, or aurora kinase inhibitor.

34. A method for treating a patient with leukemia that is resistant to at least one of dasatinib or bosutinib, said method comprising administering omacetaxine mepesuccinate to said patient.

35. The method of claim 34 wherein omacetaxine mepesuccinate is administered subcutaneously.

36. The method of claim 34 further comprising administering an additional anti-leukemia agent to said patient before, during, or after administration of omacetaxine mepesuccinate.

37. The method of claim 34 wherein omacetaxine mepesuccinate is administered for 5 days or more.

38. The method of claim 34 wherein said leukemia is also resistant to imatinib and/or nilotinib.

39. A method for treating a patient with leukemia that is resistant to dasatinib or bosutinib, said method comprising:
(a) administering omacetaxine mepesuccinate to said patient in an amount of about 1.0 to about 5.0 mg/m².

40. A method according to claim 20 or 27 wherein said administration is twice daily for seven days.

41. A method according to claim 40 wherein the dose of said omacetaxine mepesuccinate is 1.25 mg/m².

42. A method according to claim 40 wherein said omacetaxine mepesuccinate is administered for 14 days.

43. A method according to claim 41 wherein said omacetaxine mepesuccinate is administered for 14 days.
